(19) United States
(12) Patent Application Publication
(10) Pub. No.: US 2020/0275991 A1
(43) Pub. Date: Sep. 3, 2020

(54) ORAL RUBBER DAM CLAMP

(71) Applicant: CORNERSTONE SPECIALTY PRODUCTS, LLC, Irvine, CA (US)

(72) Inventors: Hamid Reza ABEDI, Irvine, CA (US); Mohammadreza FOROUGHI, Irvine, CA (US); Nick Nima KHALILI, Anaheim, CA (US)

(21) Appl. No.: 16/288,787

(22) Filed: Feb. 28, 2019

(51) Int. Cl. A61C 5/82

(52) U.S. Cl. CPC A61C 5/82; A61B 1/32

(57) ABSTRACT

A dental clamp can support a flexible sheet around a tooth during a dental operation and may include a resilient member from which jaws may attach to the tooth at opposite sides thereof—the lingual side, facing the patient's tongue, and the buccal side, facing the patient's cheek. The rubber dam clamp can be made of resin materials that may not interfere with the X-ray pattern. The rubber dam clamp can further include a tube around the clamp in order to provide suction to the patient without the need for a separate assistant to be dedicated to providing suction. The rubber dam clamp can decrease overall office costs, provide better X-ray patterns and resolution for radiographic images and retain the flexible sheet around the tooth as needed during the dental operation.

### BACKGROUND OF THE INVENTION

### 1. Field of the Invention

Embodiments of the invention relates generally to dental devices. More particularly, the invention relates to a clamp which is used by dentists as an aid for securing a so-called rubber dam sheet on the teeth, the clamp including at least a pair of gripping claws, intended to engage against a tooth neck, and a resilient bridge which connects the gripping claws. Further embodiments of the invention include a tube around the clamp in order to provide suction to the patient.

### 2. Description of Prior Art and Related Information

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

In dentistry, and especially in the field of Endodontics, it is often necessary to isolate one or more of the patient's teeth utilizing a flexible rubber dam. This isolation is important to create a clean, dry field of work which greatly enhances the likelihood of success of the operation. Usually, a metal clamp is used to retain the dam against the tooth. These metal clamps date back to the original Ivory inventions of the late 1800's and early 1900's. See, for example, U.S. Pat. No. 164,870. As confirmed by U.S. Pat. No. 4,265,623, dental dam clamps presently in use do not differ substantially from those of Ivory, either in design or in material. Often times during a dental procedure, and particularly during a root canal operation, X-ray images must be taken to monitor progress of the work. Unfortunately, the conventional metal clamp used today is often in the focal plane of the work so that its image masks a portion of the X-ray picture.

Typical clamps for holding the dam against canines or bicuspids require approximately twenty (20) pounds force to separate the clamp wings far enough apart to fit over the tooth. This force is also sufficient to hold the dam against the tooth when the clamp returns to its unseparated position. Indeed, flexural forces as low as 10-15 pounds would be adequate for this size clamp. Larger clamps, such as those used for molars, would require a greater separation force.

The above-described masking problem necessitates either taking several X-rays at different angles to accurately and completely visualize the affected area or temporarily sealing the tooth, removing the rubber dam and clamp, taking X-rays, reapplying the dam and clamp, removing the temporary filling, and proceeding with the work. Furthermore, an assistant is often used, alongside the dentist, for keeping the suction. This can increase office costs. Eliminating the dental assistant for keeping the suction would help to decrease office costs.

Moreover, with conventional dental clamps, the edges of the base of the clamp can cause gingival wounds. Further, conventional dental clamps, particularly metal clamps having sharp or pointed teeth for engaging the teeth, have a tendency to cause iatrogenic damage to the surfaces of the teeth.

In view of the foregoing, there is a need for clamp that addresses the above issues in conventional solutions.

### SUMMARY OF THE INVENTION

Embodiments of the present invention provide a dental tool comprising a tube extending through an arc shape; a first bend in each end of the arc shape of the tube, the first bend forming tube extension portions on each end thereof; a second bend at each end of the tube extension portions, the tube terminating with ends pointed away from each other; and one or more connectors attached at least to the arc shape of the tube, the connectors configured to connect the suction tube to a dental instrument.

In some embodiments, the dental instrument is an oral rubber dam clamp.

In some embodiments, the clamp is formed from a plastic material.

In some embodiments, the clamp includes an arc-shaped connector formed along a bottom portion of the tube.

In some embodiments, tooth engaging members are disposed at each end of the connector, wherein a tooth space is formed between end portions of each of the tooth engaging members.

In some embodiments, the tooth engaging members are resiliently separable to resiliently expand a distance between the end portions of each of the tooth engaging members.

Embodiments of the present invention further provide a dental tool comprising a tooth clamp including a connector having a tooth engaging member on each end thereof; a tooth space formed between facing ends of the tooth engaging members; and a tube extending from adjacent a first one of the tooth engaging members, along the connector and terminating adjacent a second one of the tooth engaging members, wherein the tooth engaging members are resiliently separable to resiliently expand the tooth space, permitting the tooth clamp to be secured to opposite sides of a tooth.

In some embodiments, the tooth clamp is formed from one or more plastic materials.

In some embodiments, the connector and at least a portion of the tube are formed in an arc shape.

In some embodiments, the tube includes a first bend in each end of the arc shape of the tube, the first bend forming tube extension portions on each end thereof, and a second bend at each end of the tube extension portions, the tube terminating with ends pointed away from each other.

In some embodiments, the suction inlet ports in at least one of the tube extension portions.

Embodiments of the present invention also provide an oral rubber dam clamp comprising a connector, extending in an arc shape, having a tooth engaging member on each end thereof; a tooth space formed between facing ends of the tooth engaging members; a tube extending from adjacent a first one of the tooth engaging members, along the connector and terminating adjacent a second one of the tooth engaging members; and one or more suction inlets formed adjacent one or both of the tooth engaging members, wherein the tooth engaging members are resiliently separable to resiliently expand the tooth space, permitting the tooth clamp to be secured to opposite sides of a tooth.

These and other features, aspects and advantages of the present invention will become better understood with reference to the following drawings, description and claims.

### BRIEF DESCRIPTION OF THE DRAWINGS

Some embodiments of the present invention are illustrated as an example and are not limited by the figures of the accompanying drawings, in which like references may indicate similar elements.

FIG. 1 illustrates a rubber dam clamp according to an exemplary embodiment of the present invention;

FIG. 2 illustrates a rubber dam clamp disposed on a patient's teeth, according to an exemplary embodiment of the present invention;

FIG. 3 illustrates a suction adaptor for use on a patient, typically used in conjunction with a conventional rubber dam clamp, according to an exemplary embodiment of the present invention and

FIG. 4 illustrates a butterfly clamp with suction according to an exemplary embodiment of the present invention.

Unless otherwise indicated illustrations in the figures are not necessarily drawn to scale.

The invention and its various embodiments can now be better understood by turning to the following detailed description wherein illustrated embodiments are described. It is to be expressly understood that the illustrated embodiments are set forth as examples and not by way of limitations on the invention as ultimately defined in the claims.

### DETAILED DESCRIPTION OF THE PREFERRED EMBODIMENTS AND BEST MODE OF INVENTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

In the following description, for purposes of explanation, numerous specific details are set forth in order to provide a thorough understanding of the present invention. It will be evident, however, to one skilled in the art that the present invention may be practiced without these specific details.

The present disclosure is to be considered as an exemplification of the invention and is not intended to limit the invention to the specific embodiments illustrated by the figures or description below.

As is well known to those skilled in the art, many careful considerations and compromises typically must be made when designing for the optimal configuration of a commercial implementation of any device, and in particular, the embodiments of the present invention. A commercial implementation in accordance with the spirit and teachings of the present invention may be configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

Broadly, embodiments of the present invention provide a dental clamp for supporting a flexible sheet around a tooth during a dental operation. The dental clamp may include a resilient member from which jaws may attach to the tooth at opposite sides thereof—the lingual side, facing the patient's tongue, and the buccal side, facing the patient's cheek. The rubber dam clamp can be made of resin materials that may not interfere with the X-ray pattern. The rubber dam clamp can further include a tube around the clamp in order to provide suction to the patient without the need for a separate assistant to be dedicated to providing suction. The rubber dam clamp can decrease overall office costs, provide better X-ray patterns and resolution for radiographic images and retain the flexible sheet around the tooth as needed during the dental operation.

The clamp according to embodiments of the present invention may be distinguished from the conventional clamps by several factors. For example, the clamp of the present invention may be constructed of a plastic material, providing a capability of resilient deformation and being permeable to X-rays. The dental clamp of plastic material, in accordance with embodiments of the present invention, gives several important advantages. For example, the clamp construction permits the production of clear and complete radiographic photographs of the anatomy of the teeth and of the position of the instruments into the channels, which permits a reduction of the number of photographs necessary for the carrying out of certain operations. The clamp construction also facilitates the production of these photographs. The clamp can be manufactured in an economic manner by a plastic molding process, thus permitting it to be discarded after use, thereby eliminating the necessity for sterilization at high temperature after use and, by using a new product for each patient, ensuring sterility of the product for the patient.

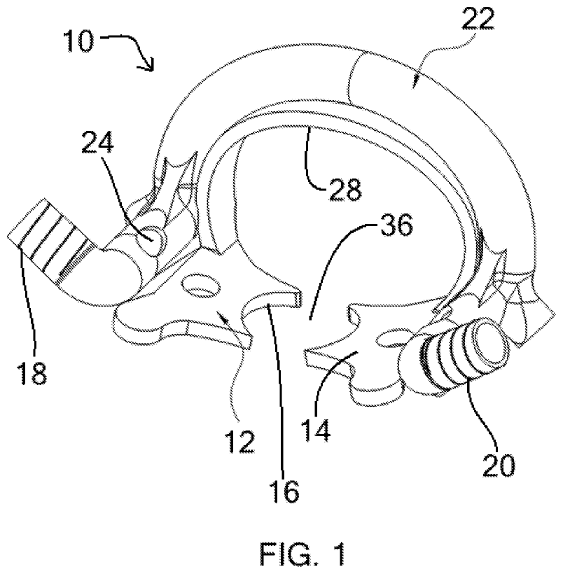

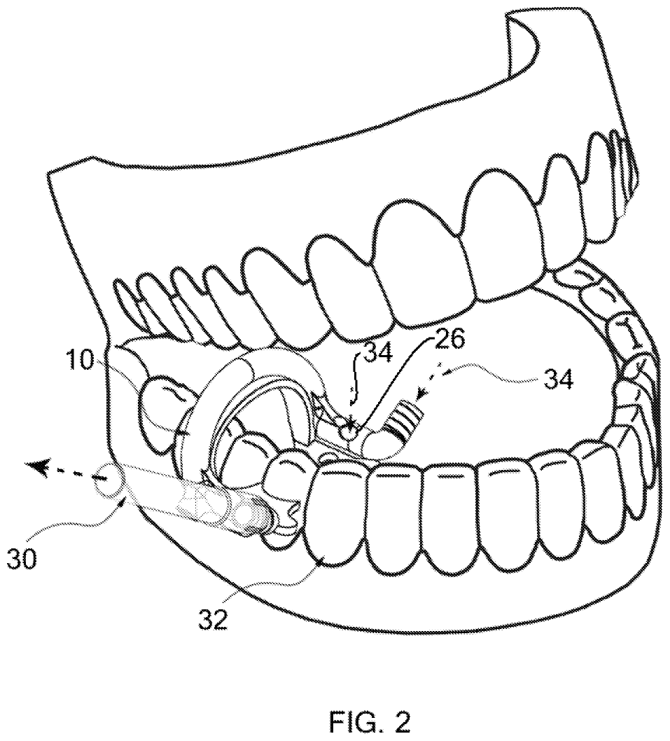

Referring to FIGS. 1 and 2, a rubber dam clamp 10, also simply referred to as clamp 10, includes tooth engaging members 12, 14 with a space 36 therebetween. The tooth engaging members 12, 14 can be connected by a resilient arched connector 28. The connector 28 may permit a user, such as a dentist, to resiliently increase the distance between the tooth engaging members 12, 14, permitting the tooth engaging members 12, 14 to clamp onto a tooth 32, as shown in FIG. 2. Typically, one of the tooth engaging members 12, 14, such as member 12, may be disposed on a buccal side of a tooth, while the opposing member 14 may be disposed on a lingual side of a tooth.

A tooth engaging surface 16 of each of the tooth engaging members 12, 14 may be shaped to engage with an outer surface of the tooth, as shown in FIG. 2. Typically, the tooth engaging surface 16 is concave and may match or be close to matching the surface of the tooth. In some embodiments, the tooth engaging surface 16 may be a resilient material that may deform to shape itself to a shape of the tooth on which the clamp is applied.

The distance between the tooth engaging members 12, 14 and the size, shape, and properties of the connector 28 may be selected so that the tooth engaging members 12, 14 may apply an appropriate amount of force to the tooth 32 to keep the clamp 10 into a tooth engaged position, as shown in FIG. 2. In some embodiments, clamps 10 may be specifically designed for engagement to different teeth. For example, one clamp 10 may be designed for engagement to molars, while another clamp 10 may be designed for engagement to canine teeth. In other embodiments, the clamp 10 may be made sufficiently resilient such that one clamp 10 may properly engage to two or more different types of teeth.

A tube 22 can extend about the connector 28 and may terminate on each side of the teeth engaging members 12, 14. In some embodiments, the tube 22 can extend about the connector 28, bend at an approximate 70-110 degree angle, typically a 90 degree angle, to extend the tube along a back side (the side opposite the tooth engaging surface 16 of the tooth engaging members 12, 14) of the teeth engaging members 12, and then may bend to extend away from the teeth engaging members 12, 14. Each end of the tube 22 may include a surface 18, 20, such as a barbed surface, for receiving a suction tube 30 thereupon. Of course, in use, the suction tube 30 may be attached to one of the surfaces 18, 20 at the end of the tube 22.

During use, water or waste materials may be sucked away from the tooth surface and the rubber dam (not shown). Suction inlet holes 24, 26 may be provided along the tube 22, typically near the tooth engaging members 12, 14 for keeping materials and water off the tooth, and inlet hole 20 may receive water and other materials from the surface of the rubber dam. Dotted lines 34 illustrate suction inlet, where the arrow exiting the suction tube 30 illustrates removal of water and/or waste to an external suction device.

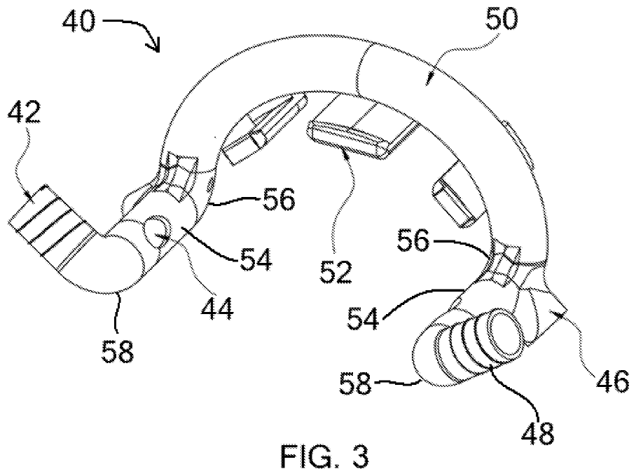

Referring now to FIG. 3, a suction device 40 can be similar to the suction tube 22, disclosed above, but instead of being attached to the connector 28 and the tooth engaging members 12, 14, the suction device 40 may be attached to a conventional clamp (not shown), or other dental apparatus, via one or more attachment members 52 to permit suction to be performed in a “hands-free” manner at multiple locations, including suction inlets 44, 46 as well as one of the suction ends 42, 48 (where the other one of the suction ends 42, 48 may be attached to a suction tube, as discussed above). The suction device 40 may be made from flexible materials, semi-flexible materials, or rigid materials, depending on the particular application.

The attachment members 52 may be designed in various manners as may be known in the art. One side of the attachment members 52 may attach to the tube 50 and another side of the attachment members 52 may include means to connect to another dental device, such as a conventional rubber dam tooth clamp. Through the use of the suction device 40, a conventional rubber dam tooth clamp may be transformed to provide the suction features provided by the clamp 10 discussed above.

In the above embodiments, the suction tube 22, 50 can extend in an arc shape, typically spanning from about 160 to about 200 degrees, with each end of the arc shape angled at first bends 56 from about 70 to about 110 degrees, typically about 90 degrees to form extension tube portions 54 that terminate with a second bend 58 such that each suction end 42, 48 extends away from each other. Typically, both halves of the tube 22, 50 are symmetrical, as shown in the Figures, however, in some embodiments, the bends may be different on each side thereof.

In some embodiments, the suction tube 22, 50 may be symmetrically formed from a left side to a right side. This allows the device to be used on either side of the patient's mouth.

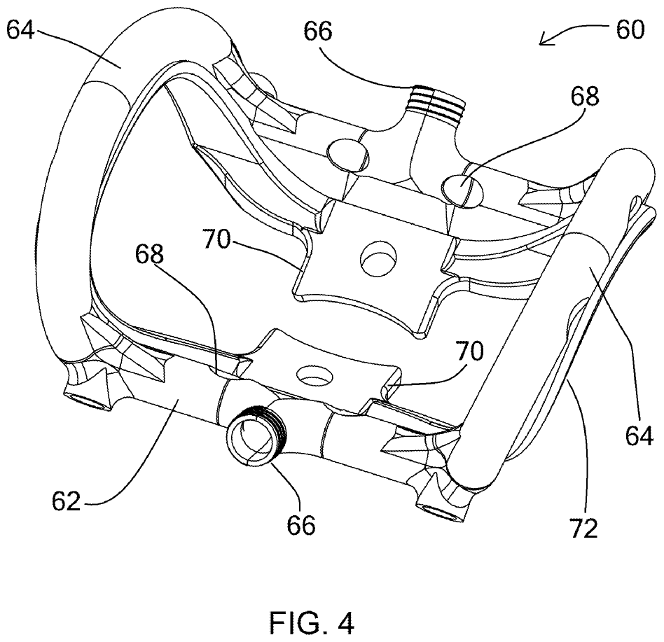

Referring now to FIG. 4, a butterfly clamp 60 with suction is shown for the front teeth application. The clamp 60 can be formed similar to the clamp 10 above, but with two arc shaped connectors with the front tooth engaging members 70 formed therebetween. More specifically, the clamp 60 can include a tube 62 that may continuously extend about first and second connectors 72. Like that described above with respect to clamp 10, the connectors 72 of clamp 60 may by arc shaped and may be shaped in a generally semi-circular arrangement on opposing sides of tooth engaging members 70. The tube 62 can include arc shaped extensions 64 that extend above a plane formed by the tooth engaging members 70.

The tube 62 can include connections 66 for attachment of a suction tube (not shown) and may further include suction inlets 68. The suction inlets 68 may be disposed at various locations along the tube. For example, the suction inlets 68 may be disposed adjacent the tooth engaging members 70.

The operation of the clamp 60 may be similar to the clamp 10 discussed above. For example, the clamp 60 may be formed from a resilient plastic material that allows the space between the tooth engaging members 70 to resiliently open to permit clamping onto a tooth.

All the features disclosed in this specification, including any accompanying abstract and drawings, may be replaced by alternative features serving the same, equivalent or similar purpose, unless expressly stated otherwise. Thus, unless expressly stated otherwise, each feature disclosed is one example only of a generic series of equivalent or similar features.

Claim elements and steps herein may have been numbered and/or lettered solely as an aid in readability and understanding. Any such numbering and lettering in itself is not intended to and should not be taken to indicate the ordering of elements and/or steps in the claims.

Many alterations and modifications may be made by those having ordinary skill in the art without departing from the spirit and scope of the invention. Therefore, it must be understood that the illustrated embodiments have been set forth only for the purposes of examples and that they should not be taken as limiting the invention as defined by the following claims. For example, notwithstanding the fact that the elements of a claim are set forth below in a certain combination, it must be expressly understood that the invention includes other combinations of fewer, more or different ones of the disclosed elements.

The words used in this specification to describe the invention and its various embodiments are to be understood not only in the sense of their commonly defined meanings, but to include by special definition in this specification the generic structure, material or acts of which they represent a single species.

The definitions of the words or elements of the following claims are, therefore, defined in this specification to not only include the combination of elements which are literally set forth. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptually equivalent, what can be obviously substituted and also what incorporates the essential idea of the invention.

What is claimed is:

1. A dental tool comprising:
a tube extending through an arc shape;
a first bend in each end of the arc shape of the tube, the first bend forming tube extension portions on each end thereof;
a second bend at each end of the tube extension portions, the tube terminating with ends pointed away from each other; and
one or more connectors attached at least to the arc shape of the tube, the connectors configured to connect the dental tube to a dental instrument.

2. The dental tool of claim 1, further comprising suction inlet holes formed at least in one of the tube extension portions.

3. The dental tool of claim 1, further comprising suction inlet holes formed in each of the tube extension portions.

4. The dental tool of claim 1, wherein the dental instrument is a tooth clamp.

5. The dental tool of claim 4, wherein the tooth clamp is an oral rubber dam clamp.

6. The dental tool of claim 4, wherein the clamp is formed from a plastic material.

7. The dental tool of claim 6, wherein the clamp includes a connector formed along a bottom portion of the tube.

8. The dental tool of claim 7, further comprising tooth engaging members at each end of the connector.

9. The dental tool of claim 8, further comprising a tooth space formed between end portions of each of the tooth engaging members.

10. The dental tool of claim 9, wherein end portions of each of the tooth engaging members, defining at least a portion of the tooth space, are formed in a concave shape.

11. The dental tool of claim of claim 9, wherein the tooth engaging members are resiliently separable to resiliently expand a distance between the end portions of each of the tooth engaging members.

12. A dental tool comprising:
a tooth clamp including a connector having a tooth engaging member on each end thereof;
a tooth space formed between facing ends of the tooth engaging members; and
a tube extending from adjacent a first one of the tooth engaging members, along the connector and terminating adjacent a second one of the tooth engaging members, wherein
the tooth engaging members are resiliently separable to resiliently expand the tooth space, permitting the tooth clamp to be secured to opposite sides of a tooth.

13. The dental tool of claim 12, wherein the dental tool is free from metal.

14. The dental tool of claim 12, wherein the tooth clamp is formed from one or more plastic materials.

15. The dental tool of claim 12, wherein the connector and at least a portion of the tube are formed in an arc shape.

16. The dental tool of claim 15, further comprising:
a first bend in each end of the arc shape of the tube, the first bend forming tube extension portions on each end thereof; and
a second bend at each end of the tube extension portions, the tube terminating with ends pointed away from each other.

17. The dental tool of claim 16, further comprising suction inlet ports in at least one of the tube extension portions.

18. An oral rubber dam clamp comprising:
a connector, extending in an arc shape, having a tooth engaging member on each end thereof;
a tooth space formed between facing ends of the tooth engaging members;
a tube extending from adjacent a first one of the tooth engaging members, along the connector and terminating adjacent a second one of the tooth engaging members; and
one or more suction inlets formed adjacent one or both of the tooth engaging members, wherein
the tooth engaging members are resiliently separable to resiliently expand the tooth space, permitting the tooth clamp to be secured to opposite sides of a tooth.

19. The oral rubber dam clamp of claim 18, further comprising:
a first bend in each end of the tube that extends along the connector, the first bend forming tube extension portions on each end thereof, the tube extension portions being adjacent each of the tooth engaging members; and
a second bend at each end of the tube extension portions, the tube terminating with ends pointed away from each other, wherein
the one or more suction inlets include a suction inlet formed in each of the tube extension portions.

20. The oral rubber dam clamp of claim 18, wherein the clamp is formed from one or more plastic materials.

21. The oral rubber dam clamp of claim 18, further comprising a second connector formed in an arc shape, the second connector extending from front tooth engaging members, the connector and the second connector extending generally in the same direction away from the space, wherein the tube extends along both the connector and the second connector.
